(19) United States
(12) Patent Application Publication
(10) Pub. No.: US 2005/0266577 A1
(43) Pub. Date: Dec. 1, 2005

(54) Ultrafiltration device for drug binding studies

(75) Inventors: John B. Lynch, Billerica, MA (US); Michele Dumon, Wasselonne (FR); Alan Weiss, Acton, MA (US); Kenneth G. Desilets, Westford, MA (US); Stephane Jean Marie Olivier, Rosheim (FR)

Correspondence Address: MILLIPORE CORPORATION, 290 CONCORD ROAD, BILLERICA, MA, 01821, US

(73) Assignee: Millipore Corporation, Billerica, MA (US)

(21) Appl. No.: 11/195,517

(22) Filed: Aug. 2, 2005

Related U.S. Application Data
(62) Division of application No. 10/456,857, filed on Jun. 6, 2003.
(60) Provisional application No. 60/386,382, filed on Jun. 6, 2002.

(51) Int. Cl. G01N 33/00

(52) U.S. Cl. 436/86

(57) ABSTRACT

The combination of a supported UF membrane having low non-specific binding (NSB) and high protein retention of the tested chemical entity (CE) in a device that is SBS complaint. The membrane is heat sealed to form one or more integral wells that are used to reduce NSB and improve protein retention and provides a simple, flexible way to reduce CE, such as drug and drug candidate (and other small molecule) NSB so that drug binding studies may more closely predict the behavior of these compounds in vivo.

### CROSS-REFERENCE TO RELATED APPLICATIONS

The present application is a divisional application of U.S. application Ser. No. 10/456,857, filed on Jun. 6, 2003, which claims the benefit of U.S. Provisional Application No. 60/386,382, filed on Jun. 6, 2002. The entire contents incorporated herewith in their entirety.

### BACKGROUND OF THE INVENTION

Protein binding is an important property for absorption, distribution, metabolism and excretion (ADME) and pre-clinical testing of chemical entities (CEs) such as drugs, drug candidates, therapeutic agents and other small molecule entities since it predicts the amount of free CE available in the plasma and/or the distribution of the CE, such as a drug, in the blood stream.

Equilibrium dialysis, a cumbersome procedure, requiring 18 to 24 hours of incubation is the accepted method today for determining CE protein binding.

More recently, the use of ultrafiltration membranes in multiwell plates has been introduced as a faster method to determine CE binding properties.

Conventionally, ultrafiltration membranes in multiple well plates have not been commercially available as the membranes are so fragile that there was no easy method for inserting them into the wells and forming a liquid tight seal between them and the well.

As an alternative, a 96 well device, known as the Microcon® 96 system, available from Millipore Corporation of Bedford, Mass., is formed of 96 individual filter devices, each having a UF membrane sealed in the filtration device by a gasket. See WO 01/05509 A1. These 96 devices are then arranged in a 96 well array (8×12) and use a receiver plate that has low non-specific binding properties to recover the filtrate. The receiver plate works well for most small molecules and drugs and represents an improvement over the prior art.

However, with low solubility or lipophilic CEs, even this device has been shown to have measurable levels of non-specific binding (NSB) for a number of low solubility and/or lipophilic CEs. In particular, the membrane in the device is sealed by an O-ring gasket to retain the membrane in the device. This gasket has a relatively high NSB. Unfortunately, this membrane is not capable of being heat sealed in place to eliminate the gasket. Moreover, as the system is formed of individual devices arranged in an array, this device has severe dimensional constraints and does not conform to industry standards (Society for Biomolecular Screening [SBS]) for dimensions for a 96 well plate device. As such, they cannot be handled by robotic laboratory equipment and are not compatible with automated high throughput screening techniques.

A true 96 well design that conforms to SBS standards, see U.S. Pat. No. 6,309,605, has allowed for UF membranes to be bonded to a plate assembly and has led to the first commercially viable UF plate. The membranes in this device are a composite UF membrane, meaning that the UF layer is formed on a pre-cast microporous membrane as the backing or support layer. The backing of the membrane is used to seal the membrane in each well. The available device uses a cellulosic UF composite membrane formed on a cast ultrahigh molecular weight polyethylene (UPE) membrane. CE non-specific binding (NSB) (the CE such as a drug candidate is absorbed or bound to the support structure and is removed from the filtrate) is very high in these devices.

An alternative membrane exists that uses a non-woven support layer in lieu of the UPE membrane. While it has lower NSB, the sealing ability of this type of membrane in a multiwell device is inconsistent and not suitable for such studies.

In order to make CE binding assays more predictive of in vivo behavior, a more universal device with low NSB, high protein retention, adequate sealing properties so that all wells are integral and that is SBS compatible is needed.

### SUMMARY OF THE INVENTION

The present invention relates to a single or multiple well device containing a UF membrane for in vivo testing of chemical entities such as drugs or potential drug candidates or therapeutic molecules. More particularly, it relates to a single or multiple well device containing a UF membrane having low non-specific binding and high protein retention for in vivo measurements such as drug-protein binding or the measurement of free drug concentration during clinical trials.

The combination of a non-woven supported UF membrane in a device to which it is heat sealed is used to reduce CE non-specific binding (NSB) and improve protein retention and provides a simple, flexible way to reduce CE, such as a drug and drug candidate (and other small molecule), NSB so that binding studies may more closely predict the behavior of these compounds in vivo.

It is an object of the present invention to provide a filtration device for performing a range of binding studies that utilizes an ultrafiltration membrane having low NSB and high protein retention.

It is another object of the present invention to provide a multi-well plate having one or more wells, each well having a bottom closed by a porous structure, said porous structure being a non-woven supported UF membrane having low NSB and high protein retention for drug binding studies.

It is a further object of the present invention to provide a filtration device comprising one or more wells, the one or more wells having a bottom with a membrane support formed therein, an ultrafiltration membrane being sealed to the membrane support of the one or more wells such that all fluid in the well must pass through the membrane before exiting the bottom of the one or more wells and the membrane having low non-specific binding and high protein retention.

It is another object of the present invention to provide a filtration device comprising one or more wells, the one or more wells having a bottom with a membrane support formed therein, an ultrafiltration membrane being sealed to the membrane support of the one or more wells such that all fluid in the well must pass through the membrane before exiting the bottom of the one or more wells and the membrane having low non-specific binding (less than 10%) and high protein retention (greater than 99%).

It is a further purpose of the present invention to provide a process for the testing of drug candidates comprising selecting a drug to be tested, selecting a testing device having one or more wells, each well having a bottom closed by a porous structure, said porous structure being a non-woven supported UF membrane having low NSB and high protein retention and being heat sealed into the wells, positioning the device over a receiver device comprised of one or more wells, each well having an open top and a closed bottom and being in register with a well of the testing device so as to receive filtrate from the one or more wells of the testing device, diluting the drug in a liquid carrier, applying the drug candidate to the one or more wells of the testing device and determining the level of drug binding of the candidate.

### IN THE DRAWINGS

FIG. 1 shows a device useful in one embodiment of the present invention in cross section.

### DETAILED DESCRIPTION OF THE INVENTION

The present invention relates to an ultrafiltration device using a membrane that is a non-woven supported UF membrane with low NSB for chemical entities, high protein retention and good sealing properties.

By chemical entity or chemical entities (CE or CEs), it is meant any low molecular weight organic compound that is a drug, an entity that has drug or therapeutic properties or is being screened for drug or therapeutic properties (also known as drug candidates).

By low non-specific binding (NSB), it is meant that the non-specific binding of the drug to the components of the device is less than about 10% loss of the drug, by mass, at low concentrations achieved during use (e.g., 10 nanomolar). By high protein retention, it is meant that the filter prevents at least 99% and in one embodiment at least 99.5% of protein from the sample fluid, such as blood plasma or serum, equal to or greater in size of the nominal molecular weight cutoff of the membrane from passing through the filter.

The present invention relates to an ultrafiltratrion (UF) membrane that is capable of being heat sealed into a filtration device, either single welled or multiple welled. The UF membrane has to have a low NSB (less than 10%) a high protein retention (greater than 99.0%) and when in the multiple well format is SBS compliant. One such membrane is known as PLGCA cellulosic membrane available from Millipore Corporation of Billerica, Mass. It is a non-woven supported UF membrane formed of a cellulosic UF layer cast on top of polypropylene non-woven material. This membrane also has low humectant levels which is helpful in drug research as the humectant often becomes an extractable in the liquid. A lower level of humectant is helpful in reducing the levels of extractables. Additionally, this membrane has a slight asymmetric pore configuration meaning that the pores on one side are smaller than the pores on the other side and there is a gradual increase in pore size from one side to another. The membrane is capable of being heat bonded to a device having one or more wells and has low NSB and high protein retention.

Additionally, other cellulose based composite UF membranes cast on a blend of polypropylene and polyethylene, on a sheath like structure comprising a core of polypropylene covered by an outer layer of polyethylene or on PTFE (polytetrafluoroethylene) resin are also useful in the present invention.

The backing itself must have low NSB, typically less than 10%. Any backing having low NSB can be used to form a composite UF membrane suitable for use in the present invention. Preferably, the backing has a pore size of less than about 80 microns on average and has as minimal a surface area as it practical while still acting as the substrate for the UF layer. Typically, a surface area of from about 0.05 about 0.5 m²/gram is preferred.

Test plates having one or more individual wells or reaction chambers are common laboratory tools. Such devices are employed for a wide variety of purposes and assays, see U.S. Pat. No. 4,902,481. These are commercially available from Millipore Corporation of Bedford, Mass. under the brand name of MULTISCREEN® plates. Single welled devices are also well known, see U.S. Pat. Nos. 3,483,768, 4,632,761 and 4,722,792. These are commercially available from Millipore Corporation of Bedford, Mass. under the brand names of CENTRICON® devices, CENTRIFREE® devices, MICROCON® devices and AMICON® ULTRA devices. While the embodiment described in detail below relates to a multiple well device, it is not meant that single well devices are excluded in any manner form the present invention.

The present invention can be made by selecting a device that has one or more wells, each well having one end open and the other end (the lower end) essentially closed except for a small opening (typically called a spout). The upper surface of the essentially closed end has an ultrafiltration membrane sealed across it such that any liquid will be retained in the well of the device until either a vacuum or positive pressure is applied to filter the liquid through the membrane. The support layer of the membrane is sealed to the upper surface of the closed end of the well by any conventional method such as heat bonding, ultrasonic bonding, vibrational bonding or friction bonding. See U.S. Pat. No. 6,309,605. It is preferred that the membrane be sealed by heat bonding. As shown in U.S. Pat. No. 6,309,605 one may use a heated die to heat the edges of the filter's support so as to cause it to melt and bond with the upper surface of the well support structure.

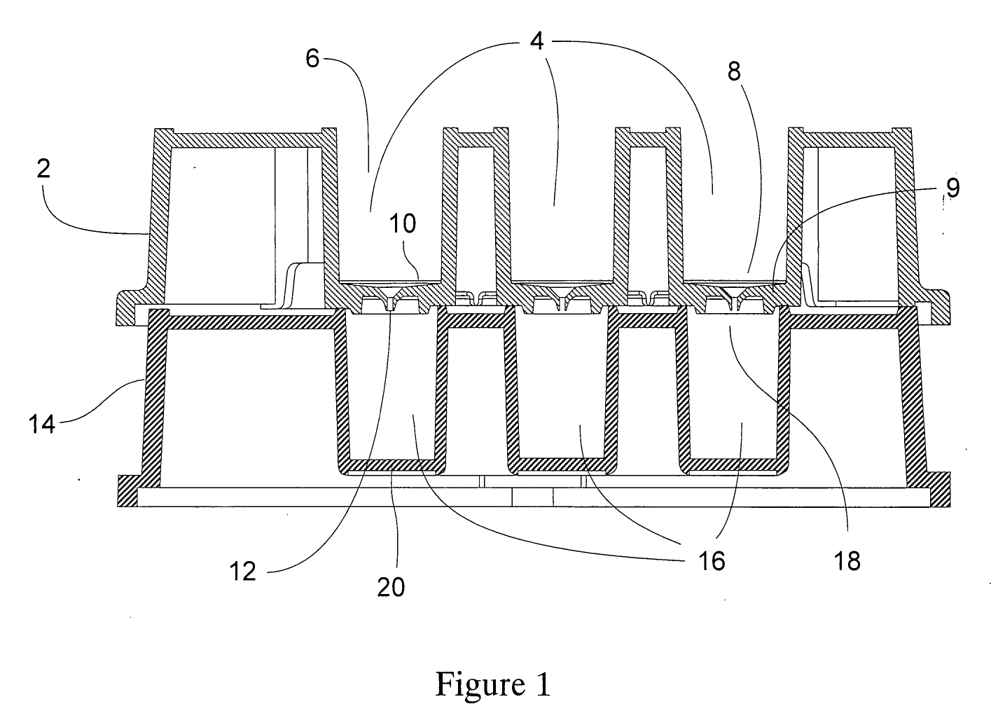

A typical multiple well device of the present invention comprises that similar to what is shown in FIG. 1. The system comprises a plate 2 which has a series of wells 4, typically 12, 24, 48 or 96 in number although lesser (such as 1,2 or 6 wells) or greater numbers (such as 384 or 1536 wells) may be used.

The tops 6 of the wells are open and the bottoms 8 are somewhat closed by a support structure 9, typically a porous web, an outer peripheral lip extending into the well, a grid of supports extending across the diameter of the well or a series of rays radiating outward from the center of the well (similar to that of a wagon wheel).

The UF membrane 10 is sealed to the top of the support structure 9 such that constituents whose size exceeds the size of the membrane's largest pore or which are retained by surface tension in the lack of a driving force for the filtration are retained within the wells and only liquid passes through the membrane 10 by either diffusion or applied pressure. An outlet 12 is formed in the well below the support 9 to allow for liquid and smaller constituents to leave the well. As shown, it also contains a director or spout 13 to concentrate or direct the exiting material to the correct location.

A receiver plate 14 is positioned below the plate 2. The receiver plate 14 has a series of wells 16 having an open top 18 and a closed bottom 20. The number of wells, their size and configuration are designed to register with those of the plate 2 such that all liquid leaving a well 4 of the plate 2 through the outlet 12 flows into a respective well 16 of the receiver plate 14.

The plate 2 is preferably made of a single piece of plastic. Two piece designs, such as an open well plate and an underdrain plate, may be used if they are sufficiently rigid to withstand the rigors of centrifugation commonly used in the filtration of serum, plasma and other viscous test fluids and generally should be permanently attached to each other by any of the well known methods including solvent bonding, adhesive bonding, vibration welding and heat sealing.

A chemical entity (CE) such as a drug candidate is diluted to a concentration believed appropriate for in vivo administration. Typically, the CE is diluted to a level of from about 10 micromolar (μM) to about 0.1 nanomolar (nM) depending on the assay and CE being tested.

The CE is then added to the open top of the wells 4 of the plate 2. After a time, typically an hour or so, the two plates 2, 14 are centrifuged, then separated and either or both the liquid in the wells 16 of the receiver plate 14 or the material on top of the filter layer in the plate 2 are analyzed for CE content.

### EXAMPLES

### Testing Membrane Non-Specific Binding

Microcon® Device Preparation:

The membrane to be tested was cut with the appropriate die cutter for the diameter of the device. The assembly consisted of placing the membrane on the support followed by addition of a gasket.

The collar is then placed on top of the assembly and sealed at a pressure varying from 65 to 100 psi.

Testing Proper Assembly and Integrity of the Device:

a) The devices were visually inspected by making sure the gasket was not deformed.

b) The devices were disassembled and checked for uniform gasket imprint on the membrane.

If not uniform, the assembly pressure was increased until upon inspection a proper imprint was observed

c) Testing of a proper seal of the device was made by adding 500 □l of “red dye” and spinning the device in a centrifuge at 14000×g for 3 minutes. Any unfiltered dye was removed from the device. The collar and gasket were removed and one looked for the absence of dye on the region of the membrane covered by the gasket. If dye was not rejected, the sealing pressure was increased. Note that a colorless ultrafiltrate was an indication that the membrane should have been pre-wetted with a suitable solvent prior to testing.

Radioactive Analyte Preparation:

In order to prepare the analyte we used the following equation:
[Concentration of Analyte]×[Volume needed for the experiment in microliter]×[Specific activity (Ci/mol)]×[1/Concentration (Ci/□l)].

If the value was less than 1 □l, the volume was increased for the experiment to reduce the pipetting error.

The required volume of analyte was added to an appropriate volume of PBS. 200 □l of this preparation was used per sample.

Membrane Testing:

Each membrane was tested in triplicate. Each Microcon® device was placed in the appropriately labeled retentate/filtrate vial. If the membrane to be tested needed to be pre-wetted before proceeding with this process, one needed to be sure that the membrane received a final rinse with PBS prior to testing. 200 □l of analyte solution was added to each Microcon® device and spun to dryness (20-30 minutes) in a microcentrifuge (14000-×g). The membranes were rinsed by adding 25 □l to each device and spun in the microcentrifuge (14000-×g) for 10 minutes.

To determine the amount of non-specific binding to the membrane, the collar and gasket were removed and the membrane placed in a glass scintillation vial. Three ml of scintillation cocktail was added to each vial. A 20 □l aliquot of the original solution was added to 3 ml of scintillation cocktail for reference. Radioactivity (in DPM) was determined by liquid scintillation counting using 1 minute counting periods and a stored quench curve as described by the equipment manufacturer.

Data Analysis:

The ratio of radioactivity found on the membrane compared to the total amount of radioactivity added was reported as a percentage and was used to assess CE NSB of the membrane being tested.

$$\% bound = ( \frac{{counts}_{membrane}}{{counts}_{total}} ) \times 100 \%$$

A first control plate, a Multiscreen® 96 well plate containing a heat sealed UPE (ultrahigh molecular weight polyethylene) composite cellulosic UF membrane, was used in a binding study. It was found to be unsuitable for that purpose as the UPE backing had high NSB (greater than 10%) and relatively high protein retention (less than 99%).

| | % Non Specific Binding at 10 nanomolar CE | | | | |
|---|---|---|---|---|---|
| | concentration in phosphate buffered saline. | | | | |
| Support | taxol | verapamil | testosterone | digoxin | warfarin |
| UPE composite | 39% | 50% | 101% | 47% | 22% |
| | 16% | 3% | 1% | 3% | 0% |
| Non-woven 2 | 8% | 5% | 1% | 2% | 1% |
| No support | 6% | 5% | 4% | 3% | 5% |

| | % Non Specific Binding at 10 nanomolar CE | | | |
|---|---|---|---|---|
| | concentration in phosphate buffered saline. | | | |
| Support | proprananol | methotrexate | ibuprofen | mannitol |
| UPE composite | 61% | 16% | 55% | 1% |
| Non-woven 1 | 0% | 0% | 0% | 2% |
| Non-woven 2 | 2% | 3% | 3% | 1% |
| No support | 4% | 4% | 4% | 2% |

A second control plate of the same materials as the first control plate had an ultrafiltrate diluent added. Little change in NSB occurred and no increase in protein retention was noted.

| | % Non Specific Binding at 10 nanomolar CE | | | | |
|---|---|---|---|---|---|
| | concentration in phosphate buffered saline. | | | | |
| Support | taxol | testosterone | digoxin | proprananol | mannitol |

| | | | | | |
|---|---|---|---|---|---|
| UPE | 64% | 80% | 92% | 70% | 2% |
| composite | | | | | |
| No support | 4% | 3% | 3% | 2% | 1% |

A plate according to the present invention having a non-woven polypropylene backed cellulosic UF membrane (PLGCA available from Millipore Corporation of Billerica, Mass.), was heat sealed into a 96 well MULTISCREEN® plate and used in a CE binding study. NSB was below 10%, protein retention was high (greater than 99.5%), all 96 wells were found to be integral and the plate was SBS compliant.

Method for Testing Protein Retention:

Device: Ultracel™ PPB 96 well filtration devices were made with two different UF membranes, PLGCA and PLGCD from Millipore Corporation of Billerica, Mass. The wells were tested for integrity using an air integrity testing. Only those wells found to be integral were used in the test.

Procedure:

Preparation of Cytochrome c:

1. 40ml of 0.25 mg/ml cytochrome c (Sigma-QC grade) was mixed in phosphate buffered saline solution (PBS).

Preparation of FITC-BSA Serum Solution:

1. 20 ml of 1 mg/ml FITC BSA (Sigma) was mixed in PBS.

2. The mix was prefilter in a stirred cell with a PLTK membrane (available from Millipore Corporation).

3. The retentate was reconstituted to the original volume with PBS.

4. The FITC BSA solution was mixed with an equal volume of clarified FBS for a final concentration of 0.5 mg/ml FITC BSA in 41 mg/ml clarified FBS.

Plate Preparation:

1. Wetted ULTRACEL™ plates with 100 μl deionized water in each well and measured integrity again. Any failures were indicated.

2. Left water in plates until ready for testing. Did not test empty wells that failed integrity test.

Test Plates and Controls:

1. Added 300 μl/well FITC-BSA serum solution to half of the cells for each plate and to 3 Centrifree® devices available from Millipore Corporation of Billerica, Mass.

2. Added 300 μl/well cytochrome c solution to the remaining wells of each plate and to 3 Centrifree® devices.

3. Put device in a Costar PS collection plate.

4. Spun all devices @ 3000×g in a swinging bucket centrifuge@ 37° C. for 30 min.

5. Measured cytochrome c in ultrafiltrate at 410 nm versus a standard curve using a Spectromax plate reader.

6. Measured volume in ultrafiltrate at 900-100 nm versus water using a Spectromax plate reader.

7. Evaluated validity of results by determining volume recovered in the plate. If the volumes were below 200 μL, then respun the plate and redid steps #5 and #6.

8. Transfered ultrafiltrates to Griner black PS plate.

9. Measured FITC BSA in ultrafiltrate via the Fluorescein method in a Wallac Victor plate reader.

Data Analysis: The FITC-BSA protein retention was calculated by the following equation:

$$\% protein\_rejection = ( 1 - \frac{{counts}_{ultrafiltrate}}{{counts}_{initial}} ) \times 100 \%$$

The cytochrome c retention was calculated by the following equation:

$$\% protein\_rejection = ( 1 - \frac{{[ cyt\_c ]}_{ultrafiltrate}}{{[ cyt\_c ]}_{initial}} ) \times 100 \%$$

Protein Retention Data

| | | | PLGCAA | PLGCD |
|---|---|---|---|---|
| Temp | Pressure | Time | Pass/Marginal | Pass/Marginal |
| 230 | 3.5 | 0.8 | 31/15 | 0/0 |
| 230 | 4 | 0.8 | 25/20 | 1/9 |
| 210 | 4 | 1.5 | 16/15 | 0/1 |
| 210 | 3.5 | 1.5 | 18/17 | 0/0 |

Only the device containing the filter of the present invention provided the desired protein retention characteristics.

The present invention provides a device and a methodology for the ADME screening of chemical entities, such a potential drug candidates or therapeutic agents that eliminates or significantly reduces the interference often found with other devices. This allows the assay to more closely mimic the actual effect that occurs in the human or animal body, allowing researchers to gain a better, faster and more accurate determination of a potential chemical entity's capabilities, allowing them to more rapidly screen through the thousands of potential candidates and eliminate those which do not have the proper characteristics and capabilities.

One particularly useful application of this technology is in drug screening using plasma, serum and other highly viscous materials as the sample fluid. Using a membrane with the required characteristics of low NSB and high protein retention in a single piece molded multiwell device format and using centrifugation as the filtration force, one is able to simultaneously process 96 samples and candidates in short order and with more accuracy.

1) A process for the testing of drug candidates comprising selecting a chemical entity to be tested, selecting a testing device having one or more wells, each well having a bottom closed by a porous structure, said porous structure being a non-woven supported ultrafiltration membrane having low non-specific binding and high protein retention and being heat sealed into the wells such that all fluid exiting the bottom of the one or more wells must pass through the membrane, positioning the device over a receiver device comprised of one or more wells, each of the one or more wells of the receiver device having an open top and a closed bottom and being in register with a well of the testing device so as to receive filtrate from the one or more wells of the testing device, applying the chemical entity in a liquid carrier to the one or more wells of the testing device, filtering the chemical entity and liquid carrier through the ultrafiltration membrane and determining the level of chemical entity binding and/or protein retention.

2) The process of claim 1 wherein the filtration is caused by applying a force to the liquid carrier selected from the group consisting of a vacuum and positive pressure.

3) The process of claim 1 wherein the membrane has a non-specific binding of less than about 10% and a high protein retention of greater than about 99%.

4) The process of claim 1 wherein the device contains 96 or more wells.

5) The process of claim 1 wherein the drug is diluted in the liquid carrier to a level of from about 10 micromoles (μM) to about 0.1 nanomoles (nM).

6) The process of claim 1 wherein the filtration is caused by positive pressure applied via centrifugation.

7) A process for the testing of drug candidates comprising selecting a chemical entity to be tested, selecting a testing device having one or more wells, each well having a bottom closed by a porous structure, said porous structure being a composite ultrafiltration membrane formed of a cellulosic ultrafiltration layer cast on top of a non-woven backing selected from the group consisting of polypropylene, a blend of polypropylene and polyethylene, a sheath of polyethylene formed over a polypropylene core and polytetrafluoroethylene, the membrane having low non-specific binding and high protein retention and being heat sealed into the wells such that all fluid exiting the bottom of the one or more wells must pass through the membrane, positioning the device over a receiver device comprised of one or more wells, each of the one or more wells of the receiver device having an open top and a closed bottom and being in register with a well of the testing device so as to receive filtrate from the one or more wells of the testing device, applying the chemical entity in a liquid carrier to the one or more wells of the testing device, filtering the chemical entity and liquid carrier through the ultrafiltration membrane and determining the level of chemical entity binding and/or protein retention.

8) The process of claim 7 wherein the membrane is a composite formed of a cellulosic ultrafiltration layer cast on top of a non-woven backing of polypropylene.

9) The process of claim 7 wherein the membrane is a composite formed of a cellulosic ultrafiltration layer cast on top of a non-woven backing of a blend of polypropylene and polyethylene.

10) The process of claim 7 wherein the membrane is a composite formed of a cellulosic ultrafiltration layer cast on top of a non-woven backing of a sheath of polyethylene formed over a polypropylene core.

11) The process of claim 7 wherein the membrane is a composite formed of a cellulosic ultrafiltration layer cast on top of a non-woven backing of polytetrafluoroethylene.

12) The process of claim 7 wherein the membrane is asymmetrical.

13) The process of claim 7 wherein the device is a single molded piece and filtration occurs by centrifugation.

14) A process for the testing of drug candidates comprising selecting a chemical entity to be tested, selecting a testing device having one or more wells, each well having a bottom closed by a porous structure, said porous structure being a composite ultrafiltration membrane formed of a cellulosic ultrafiltration layer cast on top of a non-woven backing selected from the group consisting of polypropylene, a blend of polypropylene and polyethylene, a sheath of polyethylene formed over a polypropylene core and polytetrafluoroethylene, the membrane having low non-specific binding and high protein retention and being heat sealed into the wells such that all fluid exiting the bottom of the one or more wells must pass through the membrane, positioning the device over a receiver device comprised of one or more wells, each of the one or more wells of the receiver device having an open top and a closed bottom and being in register with a well of the testing device so as to receive filtrate from the one or more wells of the testing device, applying the chemical entity in a liquid carrier to the one or more wells of the testing device, filtering the chemical entity and liquid carrier through the ultrafiltration membrane via a filtration force of centrifugation and determining the level of chemical entity binding and/or protein retention.

15) The process of claim 14 wherein the membrane is asymmetrical.

16) The process of claim 14 wherein the device is a single molded piece.

17) The process of claim 14 wherein the membrane has a non-specific binding of less than about 10% and a high protein retention of greater than about 99%.
